(19) United States
(12) Patent Application Publication
(10) Pub. No.: US 2005/0147595 A1
(43) Pub. Date: Jul. 7, 2005

(54) Bead embedded cells

(76) Inventors: Dagmar Meissner, San Diego, CA (US); Ingrid Stuiver, San Diego, CA (US)

Correspondence Address: GORDON & REES LLP, 101 WEST BROADWAY, SUITE 1600, SAN DIEGO, CA, 92101, US

(21) Appl. No.: 10/941,548

(22) Filed: Sep. 14, 2004

Related U.S. Application Data
(60) Provisional application No. 60/507,258, filed on Sep. 29, 2003.

(51) Int. Cl. A61K 45/00; A61K 9/16; A61K 9/50

(52) U.S. Cl. 424/93.7; 424/493

(57) ABSTRACT

A composition of beads formed from biologically compatible material and which hold comprise biologically active moieties. A method of treating of treating isolated biological moieties in culture media, embedding the moieties in biologically compatible material which does not incorporate adventitious excipients. A method of treating individuals in need of transplantation, which involves transplanting to the individual a composition which comprises a sufficient amount of beads.

### RELATED APPLICATIONS

This application claims priority in U.S. provisional application Ser. No. 60/507,258, filed on Oct. 1, 2003.

### FIELD OF THE INVENTION

The present invention relates to encapsulation and transplantation of cells.

### BACKGROUND OF THE INVENTION

For decades, medical researchers have worked to overcome problems of isolation, culture, storage and encapsulation of cells and tissues for transplantation from a donor to a host. Advances in the field of transplantation require reducing risk of a host immune response against an encapsulated graft, eliminating toxic components from the graft, preventing the encapsulated graft from disintegrating in vitro and in vivo, and maintaining functional viability of the grafted cells.

Alginate gel has been a widely used material for encapsulating cells for transplantation. Aqueous solutions of alginate are converted to a solid gel by treatment with a counter-ion, such as barium, strontium or calcium. Researchers typically process islet-containing, viscous alginate into alginate beads. To overcome bead instability and immunogenicity, researchers have encapsulated beads in chemical membranes.

As the alginate gel has a very wide pore size (>250,000 Daltons), it is large enough to let large immune molecules pass. Many researchers have searched for a means to isolate cells within the bead from the host's immune system. One approach was to reduce the molecular weight cutoff of the bead by wrapping the bead in a membrane using, for example, poly-L-lysine, poly-L-ornithine, polyethyleneimine, etc. This membrane unfortunately, in many cases, seemed to provide a means for cell attachment and overgrowth of these capsules, thus resulting in the failure of the graft.

U.S. Pat. No. 5,578,314 disclosed preparation of multilayer alginate beads containing a high G alginate, crosslinked with calcium as the inner core, followed by layer(s) of high M alginate crosslinked with either calcium, barium or strontium. Calcium has been considered a much safer alternative than barium or strontium for making beads. However, calcium-alginate interactions are not as strong as barium. Lanza et al. (5) has used calcium alginate beads, 3 to 4 mm in diameter, which resulted in a very large graft volume. In general, calcium has been considered too weak a counterion to produce a stable alginate gel. Calcium can be more easily displaced from the alginate gel in the presence of monovalent ions (sodium, potassium etc) or by chelators. Investigators in the field have considered calcium alginate beads not suitable for transplantation. Increased fibrosis of the graft has been observed when using CaCl₂ versus BaCl₂ (Duvivier-Kali, 2001). Barium- or strontium-containing alginate beads containing pancreatic islet cells have been successfully transplanted, yet toxicity concerns remain. There have been very few reports of processes utilizing calcium alginate beads for transplantation.

As an alternative to the alginate-PLL-alginate capsules, barium alginate beads were successfully transplanted into diabetic rodents by Zekorn et al.[1], Kloeck et al. [2], Petruzzo et al[3], and Weir et al. [4]. Yet, when ingested, barium is deposited into the muscles, lungs, and bones. Low doses act as muscle stimulants while at a higher dose, the nervous system is affected. The EPA has established a chronic and subchronic oral reference dose (RfD) of 0.07 mg/kg/day (EPA 1005a,b). Human studies identified a no-observed-adverse-effect level (NOAEL) of 0.21 mg barium/kg/day (Wones et al., 1990 and Brenniman and Levy, 1984). The barium concentration inside a graft will depend, among other factors, on the alginate composition. Based on a MIG ratio of 1.5, assuming 1 barium atom to 4 guluronic acid repeats, the maximum theoretical amount of bound barium would be approximately 4-6 mg/kg. This assumes a dose/kg of 10,000-15,000 beads with a diameter of 650 μm. No information is available on the actual barium levels or the dissociation rate of barium from various types of alginate, making the toxicity evaluation difficult.

Non-immunogenic, stable calcium alginate beads which contain viable, functional biological moieties, e.g. graft cells and which do not dissolve in media nor in the intraperitoneal cavity of the host would be desirable. For diabetes treatment, efficacy of transplantation of islet cells depends on the ability of beads to provide sufficient amounts of insulin in response to glucose stimulation, over an extended period of time, to achieve adequate glycemic control. Persistently viable biological moieties embedded in durable beads are therefore desirable.

### SUMMARY OF THE INVENTION

The invention provides a composition which includes beads formed from biologically compatible material. The beads, which comprise biologically active moieties, are substantially free of adventitious excipients. A version of the invention involves beads formed from alginate which is calcium cross-linked. In an embodiment of the beads, the biologically active moieties involve pancreatic islets.

The invention is directed, as well, to a method of treating isolated biological moieties. The method involves removing adventitious excipients from said moieties by washing the moieties in serum-free media. That is to say that the moieties are isolated from adventitious excipients before a subsequent step in which the moieties are embedded in biologically compatible material, e.g. alginate, which is substantially free of adventitious excipients to provide a bead comprising the moieties therein. The beads are cultured in media which is substantially free of adventitious excipients.

In another aspect, the invention involves a method of treating individuals in need of transplantation. The method includes the step of transplanting to the individual a composition which comprises a sufficient amount of beads of the invention. An embodiment of this method is directed to treating an individual who has diabetes with beads comprising pancreatic islet cells.

The features, objects and advantages of the invention will become apparent to those skilled in the art from the following detailed illustrations of preferred embodiments, especially when considered with the accompanying drawings, indicated by the following legends.

### DESCRIPTION OF DRAWINGS

FIG. 1 Schematic of electrostatic generator (Nisco™) used for the formation of alginate beads.

FIG. 2(a-d) are long-term graft survival curves for porcine islets encapsulated in calcium alginate beads transplanted at 10,000 islet equivalents (IE), 5,000 IE, 2,500 IE and 1,000 IE per animal in streptozotocin (STZ)-induced diabetic C57BL/6 mice. Graft survival is defined as the number of days of normoglycemia induced by the encapsulated islets transplanted in the intraperitoneal cavity.

FIG. 3 In vitro stability of calcium alginate beads when cultured in CMRL media, serum-free at 25° C., 5% CO₂.

FIG. 4 In vitro viability of islets encapsulated in calcium alginate beads, cultured in serum-free CMRL media, 25° C., 5% CO₂.

FIG. 5 Comparison of experimental islet distribution with theoretical distributions for an islet loading of 1.0 (a) and 1.2 (b).

FIG. 6 Blood glucose levels in STZ-induced diabetic NOD/SCID immunodeficient mice after transplantation of encapsulated islets using calcium alginate beads from different batches. Batch number indicated by letters in parenthesis (AK, AL, AN).

FIG. 7 Islet graft survival curves for the first-time transplantation compared to re-transplanted STZ-induced diabetic C57BL/6 mice following the initial graft failure. These data indicate a lack of sensitization of animals by the calcium alginate and/or porcine islets from the initial transplant.

FIG. 8 Summary of long-term transplantation studies in immunocompetent STZ-induced C57BL/6 mice. C57BL/6 mice were transplanted IP with islets encapsulated in alginate beads. Graft function was maintained for longer than 200 days. Animals in these studies were explanted to show the return of the diabetic state and that normoglycemia was due to porcine islet graft function.

FIGS. 9(A and B): Diabetic C57BL/6 mice were transplanted with 5K IE islets encapsulated alginate beads explanted after 257 days. Upon explantation, functional grafts exhibited very good islet viability [Live/Dead-Molecular Probes] (A), good dithizone staining and minimal cellular overgrowth (B).

### DESCRIPTION OF THE INVENTION

Unless otherwise defined, all technical and scientific terms have the same meaning as commonly understood by one of ordinary skill in the art to which this invention belongs. All publications, patent applications, patents and other reference material mentioned are incorporated by the reference. In addition, the materials, methods and examples are only illustrative and are not intended to be limiting.

### General Description and Definitions

The practice of the present invention will employ, unless otherwise indicated, conventional techniques within the skill of the art in (1) isolation and culturing cells and tissues, (2) drug delivery (3) biomaterials, (4) biochemistry; (5) molecular biology, (6) artificial organs (7) tissue engineering. Such techniques are explained fully in the literature. See, e.g. Culture of Animal Cells: A Manual of Basic Technique, 4th edition, 2000, R. Ian Freshney, Wiley Liss Publishing; Animal Cell Culture, eds. J. W. Pollard and John M. Walker; Neural Cell Culture: A Practical Approach, vol. 163, ed. James Cohen and Graham Wilkin; Maniatis et al., Molecular Cloning: A Laboratory Manual; Molecular Biology of The Cell, Bruce Alberts, et. al., 4th edition, 2002, Garland Science; Pharmaceutical Biotechnology, eds. Daan J. A. Crommelin and Robert D. Sindelar, 1997, Harwood Academic Publishers. Principles of Tissue Engineering, 1st edition, eds. Lanza et al., 1997, R. G. Lander Company; Relevant periodicals include Cell Tissue Research; Cell; Science; Nature; Biomaterials, Journal of Biomedical Materials Research, Journal of Membrane Science, Artificial Organs, Tissue Engineering.

### Definitions

The following terminology will be used in accordance with the definitions set out below in describing the present invention.

Alginate. Alginic acid is an unbranched binary copolymer of 1-4 glycosidically linked-L-guluronic acid (G) and its C-5 epimer-D-mannuronic acid (M). The proportion as well as the distribution of the two monomers determines to a large extent the physiochemical properties of alginate. The salts (and esters) of these polysaccharides are generally named alginates. It has been found that the M- and G-residues are joined together in a blockwise fashion. This implies that three types of blocks may be found, homopolymeric M-blocks (M-M-M), homopolymeric G-blocks (G-G-G) and heteropolymeric, sequentially alternating MG-blocks (G-M-G-M).

The use of alginate as an immobilizing or embedding agent in most applications rests in its ability to form heat-stable strong gels which can develop and set at room temperatures. It is the alginate gel formation with calcium ions which has been of interest in most applications. However, alginate forms gels with most di- and multivalent cations. Monovalent cations and Mg⁺² ions do not induce gelation while ions like Ba⁺² and Sr⁺² will produce stronger alginate gels than Ca⁺². The gel strength will depend upon the guluronic content and also of the average number of G-units in the G-blocks. Gelling of alginate occurs when divalent cations take part in the interchain binding between G-blocks giving rise to a three-dimensional network in the form of a gel. During gelling the concentration of divalent cations have large impact on the gel network and gel homogeneity. This may have an impact on the porosity of the gel. If no shrinking of the gel occurs, there may be more space in between the chains, leading to an increased porosity.

The sources of alginate, its mechanical properties, encapsulation mechanisms and procedures, and its biocompatibility have been well studied. Cell Encapsulation Technology and Therapeutics, edited by W. M Kühtreiber, R. P. Lanza, and W. L. Chick, Boston:Birkhäuser, 1999, p. 3-17; Fraser J E, Bickerstaff G F. Entrapment in calcium alginate, In: Bickerstaff G F, editor. Methods in Biotechnology, 1 ed. Totowa, N.J.: Humana Press Inc.; 1997. p. 61-66; 0. Smidsrød and K. I. Draget. Chemistry and physical properties of alginates. Carbohydrates in Europe 14: 6-13, 1996; G. Skjåk-Brœk and T. Espevik. Application of alginate gels in biotechnology and biomedicine. Carbohydrates in Europe 14: 19-25, 1996; Onsøyen E. Commercial applications of alginates. Carbohydrates in Europe 1996; 14: 26-31; Zimmermann, U., Cramer, H., Jork, A., Thurmer, F., Microencapsulation-Based Cell Therapy, publ. Wiley.

The term “adventitious excipients” refers to putative immunogenic materials, other than alginate (or immobilizing or embedding agents as referred to above) and donor cells, which are introduced to or incorporated into the beads. Alginate and donor cells are considered endogenous components of the beads. Accordingly, the beads of the invention are substantially free of adventitious excipients, which are of an exogenous source, typically from animal or plant or otherwise biologically derived materials or reagents in various tissue culture media and reagents. The beads of the invention do not bear adventitious excipients deposited on or otherwise adhering to them. As used herein, those biological materials other than donor cells and alginate are referred to herein as adventitious excipients. Examples of adventitious excipients excluded from tissue culture and the beads include, but are not limited to, microbial, animal or plant-derived peptides, proteins, polysaccharides.

As used herein, the term “biocompatible” refers collectively to both the bead and its contents. Specifically, it refers to the capability of the implanted intact bead and its contents to avoid detrimental effects of the body's various protective systems and remain functional for periods of at least six months. In addition to the avoidance of protective responses from the immune system, or foreign body fibrotic response, “biocompatible” also implies that no specific undesirable cytotoxic or systemic effects are caused by the bead and its contents such as would interfere with the desired functioning of the bead or its contents. The term “individual” refers to a human or an animal subject, that is, a host for the beads.

The term “bead” as used herein refers to approximately spherically shaped pieces of biologically compatible material, often generated as droplets. Methods for making beads from hydrogel materials, including alginate, are well known (e.g. Uludag, H., De Vos, P., Tresco, P. (2000) Technology of Mammalian Cell Encapsulation in Advanced Drug Delivery Reviews 42: 29-64; U.S. Pat. Nos. 6,365,385 and 6,303,355, incorporated by reference).

The term “capsule” refers to beads which are disposed in a coating or a membrane. Capsules are generally formed by first generating beads, e.g. by forming droplets of cell-containing liquid alginate followed by exposure to a solution of calcium chloride to form a solid gel. The resulting gelled spheres are coated with additional outer coatings (e.g. outer coatings, for example polylysine—see U.S. Pat. No. 6,303,355). Additional steps may involve liquefying the original bead or core gel.

The term “encapsulate” may be used herein to refer to forming a coating or membrane around a bead. In certain contexts herein, “encapsulate” refers to the process of disposing animal cells, e.g. islets, in beads, so that the cells are encapsulated within a bead.

A “biologically active moiety” is a tissue, cell, or other substance, which is capable of exerting a biologically useful effect upon the body of an individual in whom a bead of the present invention containing a biologically active moiety is implanted. Thus, the term “biologically active moiety” encompasses cells or tissues which secrete or release a biologically active molecule, product or solute; cells or tissues which provide a metabolic capability or function, or a biologically active molecule or substance such as an enzyme, trophic factor, hormone, or biological response modifier.

The term “implant,” as used herein, is defined to include all living tissues, cells, and biologically active substances intended to be implanted or transplanted into the body of an individual, as well as the act of implanting or transferring these tissues and cells into an individual. These tissues and cells include, without limitation, tissue and cells removed from a donor animal, tissue and cells obtained by incubation or cultivation of donor tissues and cells, cells obtained from viable cell lines, cells obtained from stem cells; cells obtained by genetic engineering of primary tissue, cell lines or stem cells, biologically active products of cells and tissues. Tissues may perform a useful biological function by secreting a therapeutic or trophic substance.

The term “culture” refers to 1(a) a collection of cells, tissue fragments, or an organ that is growing or being kept alive in or on a nutrient medium (i.e. culture medium); (b) any culture medium to which such living material has been added, whether or not it is still alive. 2. the practice or process of making, growing, or maintaining such a culture. 3. to grow, maintain or produce a culture.

A “cell” is the basic structural unit of all living organisms, and comprises a small, usually microscopic, discrete mass of organelle-containing cytoplasm bounded externally by a membrane and/or cell wall. Eukaryotes are cells, which contain a cell nucleus enclosed in a nuclear membrane. Prokaryotes are cells in which the genomic DNA is not enclosed by a nuclear membrane within the cells. Unless otherwise specified, the term “cells” means cells in any form, including but not limited to cells retained in tissue, cell clusters, and individually isolated cells.

“Culture medium” refers to any nutrient medium that is designed to support the growth or maintenance of a culture. Culture media are typically prepared artificially and designed for a specific type of cell, tissue, or organ.

“Tissue culture” refers to 1. the technique or process of growing or maintaining tissue cells (cell culture), whole organs (organ culture) or parts of an organ, from an animal or plant, in artificial conditions; 2. any living material grown or maintained by such a technique.

“Tissue” refers to any collection of cells that is organized to perform one or more specific function.

The term “diffusion” refers to the spontaneous mixing of one substance with another when in contact or separated by a permeable membrane or microporous barrier. The rate of diffusion is proportional to the concentration gradient of the substances, i.e. solutes and solvents, and increases with temperature. The theoretical principles are stated in Fick's laws.

The term “diffusive flux” means the velocity by which a substance moves from one point to another resulting from ambient kinetic energy derived from the immediate environment in the absence of pressure gradients (Diffusion—Mass Transfer in Fluid Systems, E. L. Cussler. ISBN 0-521-29846-6. Cambridge University Press).

The term “molecular weight cutoff” (MWCO) refers to the point or limit at which 90% of a solute is rejected from transport across a semipermeable barrier driven by a convective force or pressure gradient. It is a term used to describe the filtering or separation capability of a membrane where a fluid is flowing through a membrane driven by a pressure or osmotic gradient.

“Pancreatic Islets.” Approximately one percent of the volume of the human pancreas is made up of islets of Langerhans (hereinafter “islets”), which are scattered throughout the exocrine pancreas. Each islet comprises insulin-producing beta cells as well as glucagons-secreting alpha cells, somatostatin secreting delta cells, and pancreatic polypeptide-secreting gamma cells (PP-cells), etc. The majority of islet cells are insulin-producing beta cells. Glycemic control in diabetes has been shown to delay the onset of, and to slow the progression of associated pathological complications. A method of the invention involves transplantation of functioning pancreatic islet cells disposed in the alginate beads of the invention to diabetic subjects, to provide biological insulin replacement and to achieve normoglycemia in the treated subject for some extended period of time.

### The Invention

The invention provides calcium alginate beads which comprise mammalian cells, in particular pancreatic islets, and processes for making and using the beads. The invention overcomes obstacles associated with encapsulated islets for transplantation by:
a. Reducing risk of immune response against alginate-based grafts in which the bead is disposed in membrane made from materials such as poly-L-lysine or poly-L-ornithine;
b. Elimination of barium as a cross-linker for alginate, eliminating the toxicity concern associated with barium;
c. Instability of calcium alginate beads in vitro and in vivo.
d. Eliminating host immune response against exogenous animal excipients (serum derived biological substances) that may be deposited in or adsorbed on bead or capsule.

The invention includes method of making calcium alginate beads that remain intact in vitro and in vivo for extended periods of time while maintaining islet viability and functionality as demonstrated by normalization of blood glucose in bead-transplanted diabetic animals. The alginate beads did not dissolve in media nor in the intraperitoneal cavity. See Example Section. The beads have a mean diameter of approximately 500-700 microns, significantly reducing graft volume.

The beads, with and without islets, did not elicit any kind of immune reaction in mice. In addition, the smaller diameter of the beads reduces risk of mass or oxygen transfer limitations, as demonstrated by long-term culture studies in which a viability of 80-90% was maintained up to five weeks.

Beads that were cultured for 2 and 3 weeks were transplanted into diabetic mice and exhibited in vivo functionality. The high M alginate had a good biocompatibility as demonstrated by intraperitoneal transplantation into Lewis rats. The high M gel beads were flexible and less brittle than high G alginate, thus, the risk of bead rupture or formation of fissures was reduced.

### Method of Making Beads

Cell culture conditions applied in the preparation of the beads involved a step of removing adventitious excipients, putative immunogenic materials other than alginate and donor cells, from biological moieties and the beads. In the context of beads and capsules, alginate and donor cells are considered endogenous components of the beads. Accordingly, the beads of the invention are substantially free of adventitious excipients, which are of an exogenous source, typically from animal or plant or otherwise biologically derived materials or reagents in various tissue culture media and reagents. The beads of the invention do not bear adventitious excipients deposited on or otherwise adhering to them. As used herein, those biological materials other than donor cells and alginate are referred to herein as adventitious excipients. Examples of adventitious excipients excluded from tissue culture and the beads include, but are not limited to, microbial, animal or plant-derived peptides, proteins, polysaccharides.

The pore size of the alginate beads of the invention was greater than 250 kDa, and there was no significant limitation to the oxygen transfer at the bead size produced (500-700 microns). As a result, the need of additional oxygenation of the tissue culture medium was eliminated.

Accordingly, the beads of the present invention avoided biocompatibility issues associated with membrane-covered beads and with biologically adventitious ingredients associated with culture media. Also, the toxicity issues associated with barium or strontium when using alginate-only beads, have been eliminated.

The method of extruding viscous calcium alginate into beads can be varied. Instead of an electrostatic generator (as described below), other devices are useful for forming an alginate bead, such as a two-channel airjet, Jetcutter technology (Genia Lab, Braunschweig Germany), Piezo or coaxial air driven generators (Nisco, Zürich Switzerland).

The examples below set forth culture conditions in which all media, alginate solutions and reagent solutions were pH adjusted, isotonic, and buffered for processing islets once obtained from digested porcine pancreas.

### Method of Using the Beads of the Invention

In this invention, the beads, which comprised islets, were transplanted into individuals and provided improved treatment for diabetes without long-term immunosuppression.

The beads demonstrated improved biocompatibility compared to other membranous (e.g. polyamino acid) alginate-based encapsulation systems.

Also based on in-house data, the graft, when comparing the calcium alginate bead to a capsule similar to the Lim/Sun membranous system, has extended functionality in immunocompetent mice.

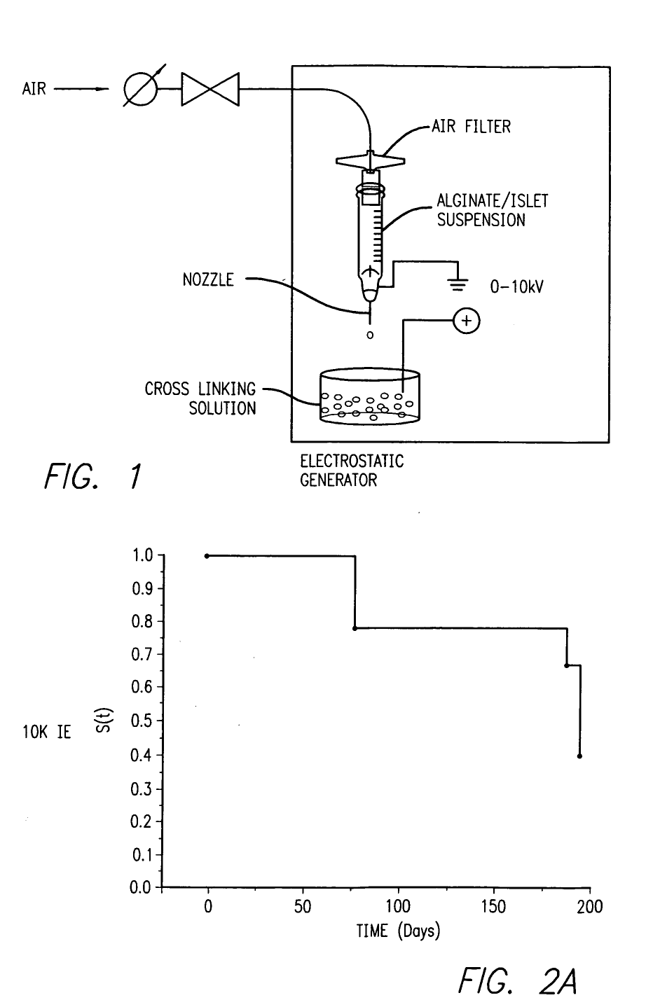

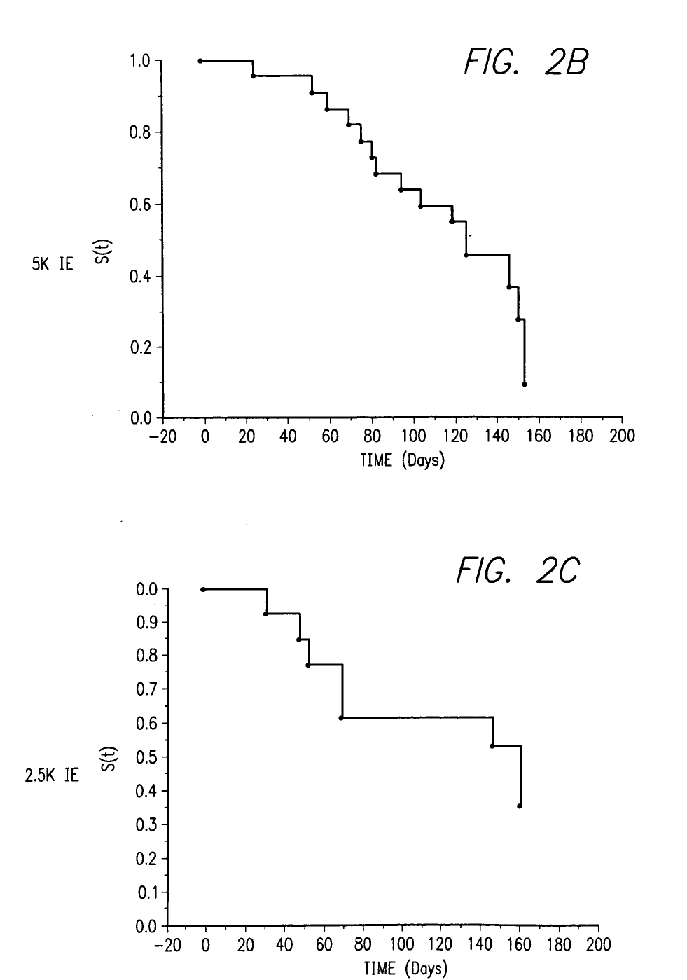

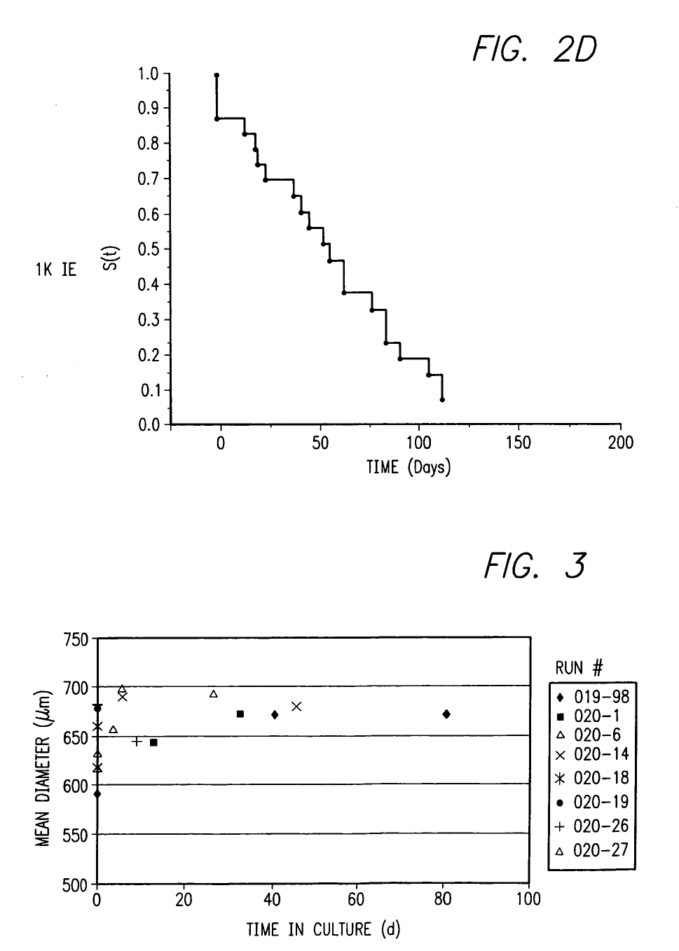

A series of experiments was performed using the fully immune competent mouse strain C57BL/6. More than 200 animals were transplanted with either islets in beads or capsules, at doses ranging from 1000 to 10,000 IE per animal. We have achieved essentially 100% successful cure of diabetes within the first week after transplant, without the use of immunosuppression. Approximately 90% of the animals transplanted with 2,500 to 5,000 IE/mouse in the bead, were normoglycemic for over 90 days however, at the 1K dose, only 25% of the animals were normoglycemic for the same period of time (FIG. 2a-d). By comparison, the capsule exhibited a strong dose response. For the capsule, 80% of the animals remained functional for 15 days at 2,500 IE/mouse and 60 days at 5,000 IE/mouse. Only at 10,000 IE/mouse, did the capsules last out to 90 days in 80% of the animals.

As the pore size of the alginate gel is very large (>250,000 Daltons) and the porosity great, there were no oxygen transfer limitations at bead size of 500-700 microns There was no need for additional means to provide oxygen to the islets in vitro or in vivo.

It was demonstrated that the islets in the beads remained viable in vitro under serum-free conditions, i.e. free of adventitious biological derived substances, for approximately 6 weeks, and showed efficacy in vivo after 2-3 weeks of culturing prior to transplantation. This demonstrated that the need for any oxygen source within the bead was obsolete. The serum-free (i.e. free of adventitious excipients) media also reduced the exposure of beads to potential antigens which, if present, could have adhered to or penetrated the beads, increasing the potential for immunogenicity in the host. The examples below demonstrated various aspects of this invention.

### EXAMPLES

### Example 1

### Encapsulation of Porcine Islets

Methods of isolating pancreatic islet cells from pigs are known in the art. Field et al., Transplantation 61: 1554 (1996); Linetsky et al., Diabetes 46: 1120 (1997). Fresh pancreatic tissue was divided by mincing, teasing, comminution and/or collagenase digestion. The islets were then separated from contaminating cells and materials by washing, filtering, centrifuging or picking procedures. Methods and apparatus for preparing islet cells are described in U.S. Pat. Nos. 5,447,863, 5,322,790, 5,273,904, and 4,868,121.

Prior to encapsulation, the porcine islets were cultured for a minimum of 12 hours in CMRL culture media (MediaTech), with 2% porcine serum, with nicotinamide, trolox (a water soluble form of vitamin E), and penicillin-streptomycin, serum-free at 25° C., 5% CO₂. The porcine serum comprises adventitious excipients.

Islets were prepared for encapsulation as follows: Islets were concentrated by simple gravity sedimentation. The islets were then isolated from adventitious excipients by washing them twice with serum free CMRL media, thereby removing the porcine serum. The islets were finally pelleted using an IEC centrifuge at a setting of “3” for 1 minute. The pellet, comprising islets (biological moieties) substantially free from adventitious excipients, was resuspended in 2% alginate (Pronova, Upsala, Sweden) in HEPES buffered saline (Sigma, St. Louis), pH 7.4, 300±30 mOsm at a concentration of 10,000-15,000 IE per mL of final alginate suspension.

Calcium alginate beads were formed using an electrostatic generator (ESG) (Nisco™, see FIG. 1), crosslinking the alginate beads in 100 mM CaCl₂ (EM Science, Gibbstown, N.J.) solution buffered with 10 mM HEPES (Sigma Aldrich, St. Louis), pH 7.4 for a minimum of 5 minutes.

The beads were then washed in serum-free CMRL culture medium and then incubated at 25° C. in 5% CO₂ until the time of transplant. The media contained 0.2 g/L of calcium chloride, which corresponded to a molar concentration of 1.8 mM.

Reagents

Purified alginate with high M content (M:G ratio of 1.5), derived from the seaweed Macrocystis pyrifera was used for these examples. This material has an endotoxin level of 50 EU/g or less. This material was produced by NovaMatrix (formerly Pronova), and is suitable for transplantation.

### Example 2

### Longevity of Beads vs. Capsules

Longevity of islets encapsulated in calcium alginate beads only (BEADS) were compared with alginate-PLO-alginate capsules (CAPSULES). When transplanted into Streptozotocin induced diabetic C57BL/6 mice, the grafts containing beads lasted significantly longer than capsules.

With calcium alginate beads, the grafts were functional for over 200 days in 40% of the animals when using a minimum dose of 2500 islet equivalent (FIGS. 2a-d). At a lower dose (1000 islet equivalents), the blood glucose levels in the diabetic mice were reduced, but were borderline normoglycemic after 50 days. By comparison, animals transplanted with islets in capsules did not sustain normoglycemia in the majority of transplants, particularly at low doses. At high doses of islets (5,000 IE per animal) in capsules, graft function was maintained for 154 days in 25% percent of the animals.

### Example 3

### Transplantation of Beads

Calcium alginate beads of the invention were stable for a minimum of 80 days in solutions containing low levels of calcium even in the presence of sodium. For example, comparing bead size, alginate beads stored in different solutions resulted in significant swelling of the beads as seen in normal saline or Hanks Balanced Salt Solution (HBSS), whereas the presence of calcium chloride prevented the swelling of the beads. Concentrations as low as 4 mM were effective (Table 1). Bead size was a good indicator of calcium displacement: When calcium was displaced by sodium, the negative charge of the alginate strands was unshielded resulting in repulsion of the individual alginate strands. This caused the gel to swell significantly.

TABLE 1
Effect of buffer composition on the diameter of
beads prepared with calcium alginate (3%).

| | time (hrs) | | |
|---|---|---|---|
| | 0 | 1 | 72 |
| Xlink | 540 | 540 | 516 |
| Saline | 540 | 717 | 701 |
| HBSS | 540 | 688 | 682 |
| CaCl2/HBSS | 540 | 540 | 511 |
| CaCl2/saline | 540 | 533 | 522 |

Diameter is given in microns.
Xlink solution contained 100 mM calcium chloride in HEPES buffer.
Saline was 0.9% saline.
HBSS = Hanks balanced Salt solution.
CaCl2/HBSS and CaCl2/saline were Hanks Balanced Salt Solution and Saline with 4 mM calcium chloride each.

### Example 4

### In Vitro Stability of Calcium Alginate Beads

The in vitro stability of calcium alginate beads in media was evaluated. For this experiment, calcium alginate beads containing islets from various batches were cultured in CMRL culture media, no serum, at 25° C., 5% CO₂. CMRL culture media had a slightly lower calcium concentration than the solutions tested in Example 2, at 0.2 g/L (1.8 mM). The media was changed 1-2 times per week, removing approximately 30% to 50% of the supernatant each time, replacing it with fresh culture media. Again, bead size was used as an indicator for bead stability. When transferring beads from crosslinking solution to media, they typically swelled almost immediately by about 40-60 μm in the presence of islets as shown in FIG. 3 (Blank beads did not exhibit the same behavior). However, the size of the islet-containing beads did not further increase over time in culture, and the beads stayed intact over the course of at least 40 days. No change of the bead surface could be detected.

### Example 5

### Viability of Beads for Extended Periods

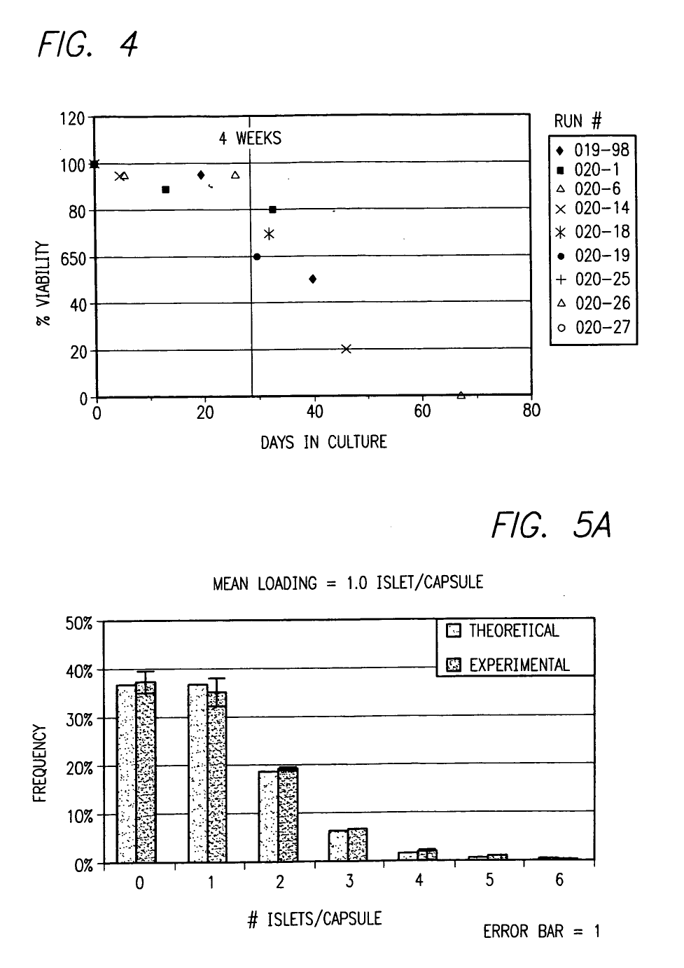

To demonstrate that the islets remained viable for extended periods of time in vitro when cultured under serum-free conditions, a stability study was conducted. Islets which were encapsulated in calcium alginate beads at various loading levels (0.6 to 2.0 IE/bead), were incubated in serum-free CMRL media at 25° C., 5% CO₂ for up to 70 days. Islet viability was monitored using the fluorescent live/dead dyes (Molecular Probes), estimating the relative ratio of live to total tissue. The islets in beads remained viable for about 4 weeks (FIG. 4). After that time, the viability dropped off fairly quickly. This drop in viability seemed to consistently occur at about the same time frame for different batches. Thus, islets were maintained for up to 4 weeks in vitro.

### Example 6

### In Vivo Viability of Encapsulated Islets

In vivo functionality for beaded islets which were previously cultured for up to 3 weeks was demonstrated. Islets in calcium alginate beads, were cultured at 25° C., 5% CO₂ in serum-free media and then transplanted into STZ-treated mice (1000 IE/mouse). These animals were normoglycemic within 1 week of transplantation, and maintained normoglycemia for up to 50 days (data not shown here). This suggests that prolonged culture of islets in the beads of the invention provided a functional graft.

### Example 7

### Stability of Islets in Calcium Alginate Beads

In vivo experiments were conducted to evaluate the stability of islets in calcium alginate beads. For that purpose, two series of studies were initiated, testing the system first in immunodeficient and then immunocompetent mice which were made diabetic by STZ injection. In the first series of studies (AK, AL, AN) NOD/SCID mice were transplanted with calcium alginate beads containing 4,000 to 10,000 islet equivalents intraperitoneally. These animals were normoglycemic for a minimum of 150 days suggesting that the graft was functional, the islets were viable (FIG. 6).

Beads from three animals of the “AN” group were explanted and analyzed. The bead size of the explanted beads was the same as the beads prior to the transplantation (Table 2), suggesting that the beads were stable in vivo. Based on the extended functionality of the islets in vivo, these data suggested that the need for any oxygen generating component within the bead system was not necessary.

As additional proof of the stability of the alginate beads, studies in immunocompetent mice (C57BL/6) were initiated as previously shown in FIG. 2. We have seen graft function for up to 180 days and longer in these animals suggesting again that the graft is still functional and viable.

TABLE 2
Size of explanted beads after 35 days in the peritoneum of 4
NOD/SCID mice compared to the bead size prior to transplantation.

| SAMPLE ID | pre TX | AJ1 | AJ2 | AJ11 | AJ12 | avg (explanted) |
|---|---|---|---|---|---|---|
| diameter (μm) | 686.8 | 687.0 | 670.7 | 673.9 | 668.4 | 675.0 |
| SD (μm) | 15.2 | 12.5 | 29.3 | 27.7 | 29.8 | 24.83 |

### Example 8

### Loading Islets into Beads

Loading of islets within the beads has been improved. In the past, researchers have used very low levels of islets when preparing the alginate beads. Typical levels were around 900-3,000 islets/mL of alginate, leaving as many as 30% of the beads empty. To reduce the number of “empty” beads, the loading of islets within the beads was increased. The process used approximately 10,000-15,000 islet equivalent per mL of suspension.

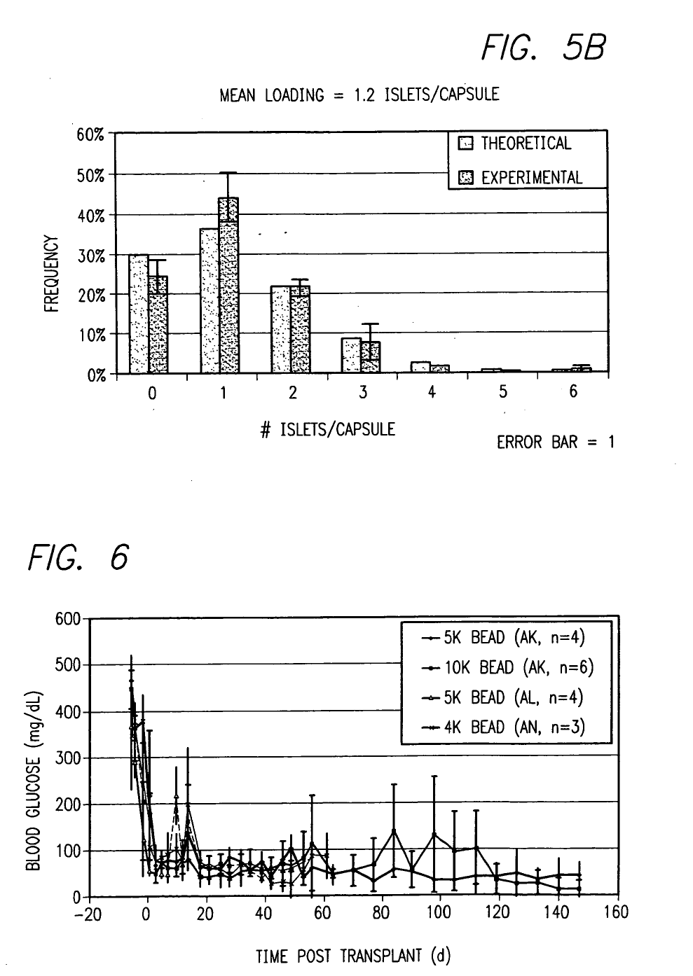

To determine if the distribution of islets per capsule and the frequency of blanks correlated to the statistical distribution or if it was due to non-homogeneous suspension of islets in the alginate or settling of islets during the ESG run, the experimental data was compared to a statistical model. For that purpose, the Poisson distribution was utilized to determine the theoretical distribution for a given average islet loading:
P(x)=(e^{-λ}*λ^{x})/x!
with P(X) being the probability of having 0, 1, 2, . . . islets in a capsule and λ being the mean islet loading over a population of capsules. The comparison of the theoretical distributions derived from this model with experimental data (FIG. 5) for two different islet loadings (1.0 and 1.2 islets/capsule) suggested that the results were very similar to the statistical probability. Thus, the islets were homogeneous in the alginate and settling was not a problem. The occurrence of blank capsules as a function of islet loading compared favorably to the statistics as well.

Pharmaceutical preparations of the alginate in beads or other forms are formulated according to well known methods as found in preparation for example for wound healing and ulcer treatments (LAM Pharmaceutical, 3M™ Tetagen™) or cell encapsulation. Please refer to company websites from Nisco Engineering, ISP, FMC Biopolymer/Novamatrix, or Instech for additional information.

### Example 9

### Sensitization Against the Graft

We tested for evidence of immunological sensitization of the mice to the calcium alginate beads and/or islets. After graft failure, animals were re-transplanted with 2,500 IE. The preliminary data indicate that there is no sensitization occurring in mice to porcine islets encapsulated in alginate bead (FIG. 7). Animals transplanted with fresh encapsulated islets exhibited graft failure 5-15 days post transplant whereas animals re-transplanted with the same dose exhibited graft function for 20-45 days after the re-transplantation. There was no deleterious effect on graft survival upon a second exposure to encapsulated islets, despite the fact that the first islet transplant had failed. These results are consistent with the conclusion that the immunoisolation of pig islets created by the alginate bead is sufficient to prevent immune sensitization by even a failed transplant.

### Example 10

### Explantation Experiments

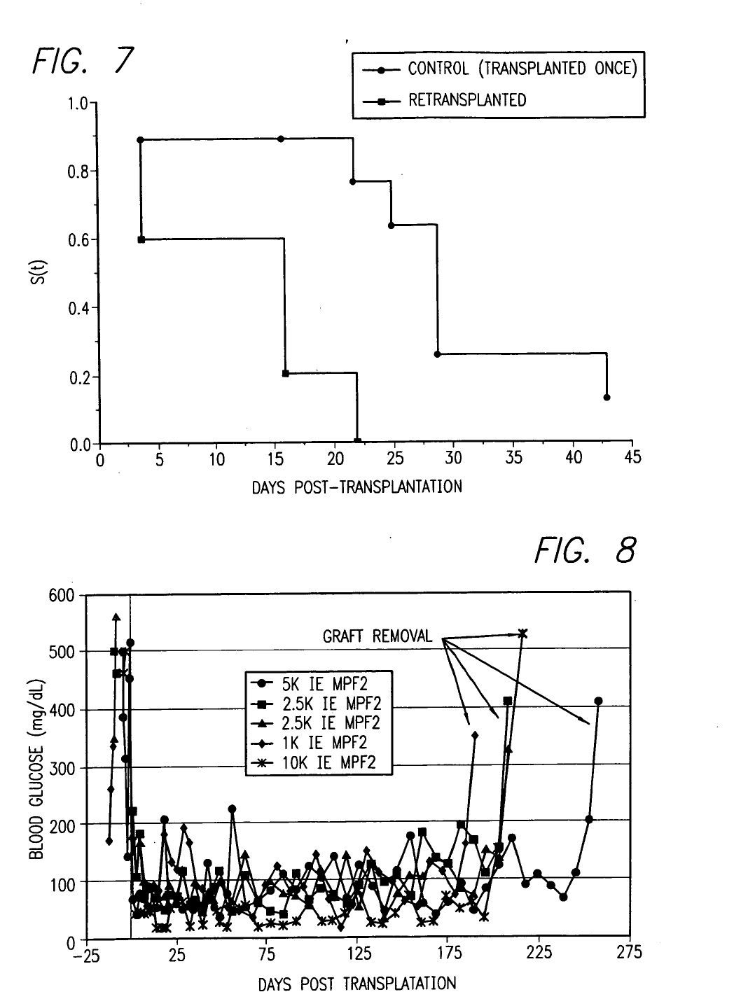

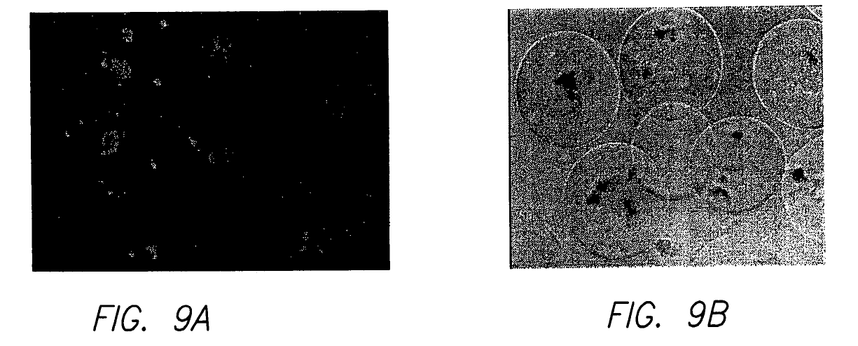

Immunocompetent diabetic C57BL/6 mice which were previously transplanted with 2,500 to 10,000 IE/animal were included in an explantation study using the methods described above. After 180 to 250 days in vivo, the beads were recovered to verify that the sustained normoglycemia was due to graft islet function. Post-explantation, the animals returned to hyperglycemia (FIG. 8). In addition, the explanted beads were essentially free of cellular overgrowth, and the islets were viable and stained positive for insulin (FIGS. 9A and B).

Other types of alginate with a wide range of M:G ratios are useful as material for forming the beads of the invention. Other biologically compatible materials useful for forming beads of the invention include polyelectrolyte systems, including agarose, and thermoplastic polymers as described in Uludag, H., De Vos, P., Tresco, P. (2000) Technology of Mammalian Cell Encapsulation in Advanced Drug Delivery Reviews 42: 29-64.

This invention is not restricted to beads which contain islets. The scope of the invention includes beads which comprise biologically active moieties.

The applications of the present invention are not restricted to treating diabetes. Bead encapsulation of appropriate biological moieties finds use in these non-limiting examples: treatment of hemophilia B, growth hormone for treatment of dwarfism, treatment of kidney failure (urea removal), treatment of liver failure, interferon production, treatment of Parkinson's disease, treatment of hypocalcemia, treatment of myocardial infarction, treatment of chronic pain.

Apart from transplantation of the beads of the invention, other applications include large scale production of cell-derived molecules, clonal selection of desired cell phenotypes, in vitro culture of cells dependent on close cell-cell contact, in vivo cell culture, reproductive technology, and cytotoxicity testing (See Uludag, H., et al., ibid, p. 31).

1. A composition comprising beads formed from biologically compatible material, said beads comprising a biologically active moieties, said beads substantially free of adventitious excipients.

2. The composition of claim 1 wherein said biologically compatible material is alginate.

3. The composition of claim 2 wherein said alginate is calcium cross-linked.

4. The composition of claim 1 wherein said biologically active moieties comprise pancreatic islets.

5. The composition of claim 1 wherein the diameter of said beads is less than 1 mm.

6. A method of treating isolated biological moieties comprising the steps of
a. isolating said moieties from adventitious excipients;
b. embedding said moieties in biologically compatible material substantially free of adventitious excipients to provide a bead comprising moieties therein; and
c. culturing said beads in media which is substantially free of adventitious excipients.

7. The method of claim 6 wherein said biologically compatible material is alginate.

8. The method of claim 7 wherein said alginate is calcium cross-linked.

9. The method of claim 6 wherein said biologically active moieties comprise pancreatic islets.

10. The method of claim 6 wherein the diameter of said beads is less than 1 mm.

11. A method of treating individuals in need of transplantation, comprising the step of transplanting to said individual a composition comprising a sufficient amount of beads formed from biologically compatible material, said beads comprising biologically active moieties, said beads substantially free of adventitious excipients.

12. The method of claim 11 wherein said individual has diabetes.

13. The method of claim 11 wherein said biologically compatible material is alginate.

14. The method of claim 11 wherein said biologically active moieties comprise pancreatic islets.
